(19) United States
(12) Patent Application Publication
(10) Pub. No.: US 2013/0253371 A1
(43) Pub. Date: Sep. 26, 2013

(54) Surgical Instrument for Deep Tissue and/or Cell Sampling

(71) Applicant: Rafic Saleh, Aguadilla, PR (US)

(72) Inventor: Rafic Saleh, Aguadilla, PR (US)

(21) Appl. No.: 13/427,811

(22) Filed: Mar. 22, 2012

(51) Int. Cl. A61B 10/02; A61B 10/06

(52) U.S. Cl. 600/569; 600/562

(57) ABSTRACT

A tissue and/or cell sampling device for biopsies having a catheter sized for translation through a lumen on an instrument such as a bronchoscope. An aspiration needle engaged to a control member through the catheter is controllable to remove a core of tissue from a sampling location. Forceps or a brush are translatably engaged within the axial core of the needle and are translatable from the tip of the needle to take tissue and/or cell samples. The device is employable to allow the surgeon to use either sampling device independently, or sequently to allow deep tissue and/or cell samples to be retrieved from the distal end of the core tissue sample retrieved by the needle.

### BACKGROUND OF THE INVENTION

1. Field of the Invention

The present invention relates generally to surgical instruments employed in a biopsy of patient tissue. More specifically, the invention relates to a multipurpose sampling device having a combination aspiration needle and biopsy forceps, or combination aspiration needle and cytology brush. The device is especially well configured for taking deeper tissue and/or cell samples than is currently possible through the provision of a coring needle to form a tunnel into tissue whereafter the forceps or cytology brush may be employed to take tissue and/or cell samples which are protected by the surrounding needle during retrieval.

2. Prior Art

Screening and early detection, diagnosing, and subsequent treatment of cancer can be a key step in fighting the disease before there are any noticeable symptoms. A commonly known method for screening a patient for cancer involves taking a body image via magnetic resonance imaging (MRI), x-ray, or other imaging method which provides the physician with means to search for visual cues to potential disease. However, there is much speculation on the benefits of performing conventional imaging screening techniques.

For example, there is a possibility that radiation exposure during x-ray imaging can pose the risk of initiating a new cancer in a healthy person. Further, typical screening methods are not necessarily useful for many cancers and the possibility of a false positive or false negative diagnosis is widely known where electronic imaging of soft and hard tissue is employed for such a diagnosis. Consequently, such tests are overly dependent on the skill of the medical professional performing the test and visual analysis of the image by a radiologist or imaging specialist. Thus, the diagnosis can be highly subjective which can cause a misdiagnosis absent a subsequent tissue and/or cell sample from the area of suspected anomaly.

Subsequent to imaging, once a potential cancer is suspected, or thought to be found, the patient is subjected to invasive diagnostic follow-up procedures to ascertain the validity of the radiological diagnosis. Such procedures conventionally involve a surgical procedure where a tissue and/or cell sample from the suspected disease site is retrieved. Commonly known as a biopsy, subsequent to capture of an appropriate tissue and/or cell sample, the sampled tissue is visually and/or chemically analyzed.

An excisional biopsy occurs when a surgeon removes an entire lump or area of tissue. A more common procedure where the potential abnormal tissue is buried in a vital organ such as the lungs, is a surgical procedure known as an incisional biopsy or core biopsy. Another sampling procedure employed by surgeons is an aspiration biopsy where a sample of tissue or fluid is retrieved and removed using a needle adapted for such.

As can be imagined, with the subjective nature of electronic imaging, especially in deep body tissues, concise and accurate diagnostic capability provided by the actual retrieval of tissue, yields much more conclusive results. When dealing with a potential life threatening disease, the physical confirmation of tissue suspected of abnormality can be extremely beneficial.

However, a challenge to the surgeon when performing a biopsy exists due to the nature of the procedure. This is especially true where samples of tissue buried deep in the body or an organ are required as it can be very difficult to remove the correct type of tissue and/or cell sample from the preferred location. This occurs even if the location has already been electronically imaged.

Modernly, there are electronic navigation and real time imaging instruments available to help guide the surgeon to the proper tissue and/or cell sample site within the patient's body. During this process, surgeons generally employ sampling surgical instruments which are operationally engaged to the distal end of a catheter, which may take time and patience to properly position. Positioning is accomplished using a camera at the end of a scope instrument having internal lumens, and/or using a triangulating radio frequency (RF) system which matches a virtual image of the patient with a current location of the distal end of the scope or catheter to position the sampling component correctly.

Once properly positioned, a different problem can occur since the instrument being navigated generally follows a body lumen or hollow cavity of the body to reach the point of sampling. At the sampling point, currently, the tissue and/or cell sample is taken from the tissue in front of the camera or at the surface of the tissue located using RF navigation. This, however, is not the most desirable tissue and/or cell sample in many instances since the suspected cancerous or other diseased tissue lies well beyond the surface layer of the body tissue at the point of sampling.

Currently, a coring needle may be employed to sample a small core of tissue at the sampling point which descends into the organ being sampled at partial lengths of the needle. Alternatively, a biopsy forceps or a cytology brush may be employed, again to slice a sample of the tissue at an exterior layer of the organ or body tissue in question.

Sampling tissue deeper within the organ or tissue is a problem, however. Forceps and cytology brushes are designed for surface sampling or sampling a small distance below a surface layer of the organ or tissue sampling site. Coring needles are limited by the short length of the core itself to a depth below the organ or tissue sampling site the needle length determines. Sampling deeper is not easily accomplished, if it is accomplished at all.

Even if deeper samples are not desired, another problem exists for the surgeon should a different sampling surgical instrument be required than the one mounted on the distal end of the catheter traversing the scope lumen. Changing the sampling surgical instrument requires the removal of the controlling catheter and re-navigation to the sampling site, which not only requires valuable surgeon time, it multiplies the risk to the patient from continued anesthesia and potential injury from the catheter travel.

For example, biopsy forceps are a hinged instrument having jaws or a grasping end which are adapted during closure to cut and capture substantial tissue and/or cell samples. However, conventional forceps designs are only intended for tissue capture at the first or surface layer of tissue, and consequently not generally employed for deeper tissue and/or cell samples such as in the lower lobes of the lung. If the surgeon wishes a slightly deeper sample, the control catheter must be removed and a needle aspiration device substituted.

Needle aspiration or fine needle aspiration (FNA) employs a coring needle which is adapted for sampling of tissue at deeper organ and body positions. However, the tissue or fluid sample removed, is generally small in quantity and the depth is as noted limited by the coring wall. For example, the surgeon can navigate such a needle deep in the lungs to positions up to 16 mm using such a sampling needle operatively engaged to a catheter. However, once the needle sampling instrument has been threaded to the sampling position, the small samples retrieved may not accurately represent the surrounding diagnosed abnormal tissue area. Further, during retraction, they can become mixed with other tissue and the small samples of larger surrounding identified tissue retrieved, frequently yield a false negative diagnosis which could have been avoided with larger samples.

However, to achieve a larger sample with a biopsy forceps, if it is determined the instrument could be navigated to the desired position, again requires removal of the first instrument. Thereafter the forceps are navigated to the proper position at the expense of time and further exposure of the patient to a second threading of a second instrument to the position where it has been determined forceps may be employed instead of a coring needle.

As such, there is a continuing unmet need for an improved medical tissue sampling instrument. Such a device should provide the beneficial qualities of both an aspiration needle and a biopsy forceps, and provide a means to retrieve deep tissue and/or cell samples from a sampling site. Such a device should provide a means to protect the integrity of the sample during retrieval in both size and type of tissue. Such a device should also provide the user the concurrent option to employ either or both a biopsy forceps or needle aspiration should surface level or simple tissue sampling be determined acceptable once proper positioning within the body of the patient is determined.

Such a device being a combination aspiration needle and biopsy forceps, should provide the surgeon the option of capturing substantial sized deeply located tissue and/or cell samples from the sampling site if possible, and to also capture samples with an aspiration needle should such be better employed at the sampling site. The combination of both instruments in addition to providing options to the surgeon for use of both, also limits the exposure of the patient to the potential for multiple catheter engagements and removals which heretofore are required to change instruments.

### SUMMARY OF THE INVENTION

The surgical instrument device herein disclosed and described provides a solution to the shortcomings noted in prior art. The disclosed device remedies the above noted problems with conventional tissue sampling at a determined sampling location through the provision of a combination aspiration needle and secondary tissue sampling means such as a biopsy forceps or cytology brush, both of which are operationally engaged to the distal end of a single catheter configured for translation through a camera bearing scope or other lumen bearing instrument. The control catheter for the device is configured with appropriate lumens and control wires for operative control and/or translating sheath type operation.

In a particularly preferred mode, the device provides a combined aspiration needle and biopsy forceps having tissue sampling jaws as a tissue sampling means at the determined sampling site in a patient. In accordance with the current preferred mode, the device is operationally configured to employ a biopsy forceps handle at a proximal end, which communicates along a guide shaft or control wire to hinged jaws at the distal end of the catheter or other lumen bearing conduit. An aspiration needle is additionally disposed at the distal end of the catheter and so positioned is coaxially and telescopically engaged to surround the hinged forceps, preferably at or near the jaws or grasping end of the forceps. A sleeve of teflon or other suitable polymeric material preferably engages over the forceps and needle as a protective sleeve.

In use the biopsy forceps are translatable within the sleeve to extend from the distal end of the surrounding aspiration needle. This translation allows a portion of the distal end of the forceps including the jaws or grasping end, to be moved to a retracted position protected within the core of the aspiration needle, or translated to an extended position where a the jaws of the biopsy forceps are translated to project past the open distal end of the aspiration needle where a deep tissue and/or cell sample may be taken and translated back inside the protected confines of the aspiration needle.

During operative employment of the device, camera aided navigation or RF aided navigation or combinations thereof are employed to navigate a camera bearing scope or catheter to a sampling site. Once the device engaged to the distal end of a control catheter is navigated through patient tissues to the sampling site, sampling may take place.

For shallow or surface tissue and/or cell samples, either the aspiration needle or the forceps may be employed at the discretion of the surgeon. However, the device provides a means to retrieve and protect deep tissue and/or cell samples from the sampling site also through the provision of the coaxially translatable forceps engaged within the axial passage of the aspiration needle.

In a deep tissue sampling process, once the device is properly positioned using RF or camera aided navigation, from the distal end of the control catheter, the aspiration needle is inserted into the organ or tissue sampling site, for a depth, for example of 5-25 mm. Once the aspiration needle is engaged at the desired position and depth, deeper within the organ or tissue sampling site, the jaws of the biopsy forceps may be translated to an extended position, projecting a distance past the distal end of the aspiration needle, and operated by control wire, to thereby provide a means to capture a large desired tissue and/or cell sample, from a deep position at the identified tissue sampling site. Operation of the forceps handle engaged to a control wire communicating with the biopsy forceps, and with translation of the lumen bearing catheter or sheath, allows the physician to translate the forceps to the extended or retracted position, and to manipulate the jaws to slice a large sample of deeply positioned tissue as needed.

Once the larger sample is taken using the biopsy forceps, they may be retracted back into the axial passage of the aspiration needle, where both the forceps and the tissue and/or cell sample are surrounded and protected from tissue contamination or loss during removal from the organ or tissue sampling site. Upon removal of the aspiration needle from the tissue sampling site, it may be translated along with the catheter to a position outside the patient, all the while with the forceps and sample being protected from possible loss or contamination during removal translation of the catheter.

In accordance with a particularly preferred mode, the present invention provides a medical instrument device with great utility in that it advantageously combines biopsy forceps and an aspiration needle operationally positioned at the distal end of a single lumen of a catheter. The device allows physicians to employ conventional camera aided or RF aided positioning, to translate the lumen bearing the device, to the sampling site in the body. Once so positioned, the surgeon may take very deep tissue and/or cell samples using the aspiration needle to tunnel into the sampling site and forceps to retrieve a larger sample. Should deep tissue sampling not be desired, the surgeon is provided with two different sampling instruments to choose from for a shallow tissue sampling. Such a combination thus is highly utilitarian since it provides the surgeon with a choice of a deep tissue sampling or multiple shallow tissue sampling components, without the need for time consuming removal and repositioning and risk of same to the patient.

In another particularly preferred mode of the invention, the tissue sampling means employed in combination with the aspiration needle is a coaxially engaged cytology brush. In use the operationally positioned cytology brush communicates with a control or handle at the proximal end of a catheter and can be telescopically translated from a retracted position surrounded by and within the axial core of the aspiration needle, to an extended position, projecting from the distal end of the aspiration needle as needed. Samples taken by the cytology brush are also protected during retrieval from damage, loss, or contamination, by the surrounding wall of the aspiration needle once the brush is translated back therein.

The device may also be provided with a single aspiration needle, and a kit bearing the biopsy forceps and cytology brush which allows the surgeon to choose which translating instrument to engage with the aspiration needle for a procedure. Engagement would be cooperative fasteners such as a threaded receiver and cooperatively threaded member for engagement therein.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and mayor may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and mayor may not be present depending upon whether or not they affect the activity or action of the listed elements.

It is an object of the invention to provide a combination biopsy forceps and aspiration needle configured to allow physicians or other medical professionals to employ both instruments in a progression to obtain deep tissue and/or cell samples from identified tissue and/or cell sample sites of potentially abnormal tissue in a patient.

It is an object of the disclosed device having a combination biopsy forceps and aspiration needle device to provide more accurate tissue sampling within a desired area of tissue by providing the ability to secure larger tissue and/or cell samples and protect the retrieved sample during translation of the device from the body.

It is another object of the invention to provide a combination aspiration needle and cytology brush or biopsy forceps to provide the medical professional options as to which of two components to employ for a shallow tissue and/or cell sample, after a single positioning of a catheter operationally engaged thereto.

These together with other objects and advantages which become subsequently apparent, reside in the details of the construction and operation as herein described with reference being had to the accompanying drawings forming a part thereof, wherein like numerals refer to like parts throughout.

### BRIEF DESCRIPTION OF DRAWING FIGURES

FIG. 1 shows a disassembled view of a particularly preferred mode of the device depicting a forceps with control handle, needle component, and protective sleeve.

FIG. 2 is a close up view of the distal end of the forceps detailing the forceps jaws used for grasping and removing larger tissue and/or cell samples.

FIG. 3 shows a view of yet another particularly preferred mode of the invention wherein a cytology brush is employed as a means for deep tissue sampling.

FIG. 4 depicts an assembled view of the preferred mode of the device of FIG. 1 with the forceps and needle in a stored or retracted position.

FIG. 5 depicts an assembled view of one preferred mode of the device of FIG. 1 with the needle in an extended position and forceps in a stored or retracted position protected during insertion or retraction from the patient.

FIG. 6 depicts an assembled view of the preferred mode of the device of FIG. 1 with the forceps and needle in the as-used tissue sampling position.

FIG. 7 shows a close up view of the forceps in the extended or as-used position with the jaws opened as needed for grasping tissue for a deep tissue and/or cell sample or surface sample.

FIG. 8 depicts the device in the position of FIG. 5.

FIG. 8a depicts the biopsy forceps in the retracted position coaxial with the aspiration needle.

FIGS. 9a-9c depict the device translating within a lumen on a camera bearing or RF positioning-enabled bronchoscope as employed for a lung tissue biopsy.

FIG. 10 depicts components forming an inner tubular structure which translates within a surrounding outer tubular structure.

FIG. 11 depicts a translation of the inner tubular structure within the coaxial outer tubular structure to translate the needle between a retracted position and extended position.

FIG. 12 depicts the inner tubular structure translated to the extended position and the translation of a control wire to translate the engaged forceps to the extended position.

### DETAILED DESCRIPTION OF THE PREFERRED EMBODIMENTS OF THE INVENTION

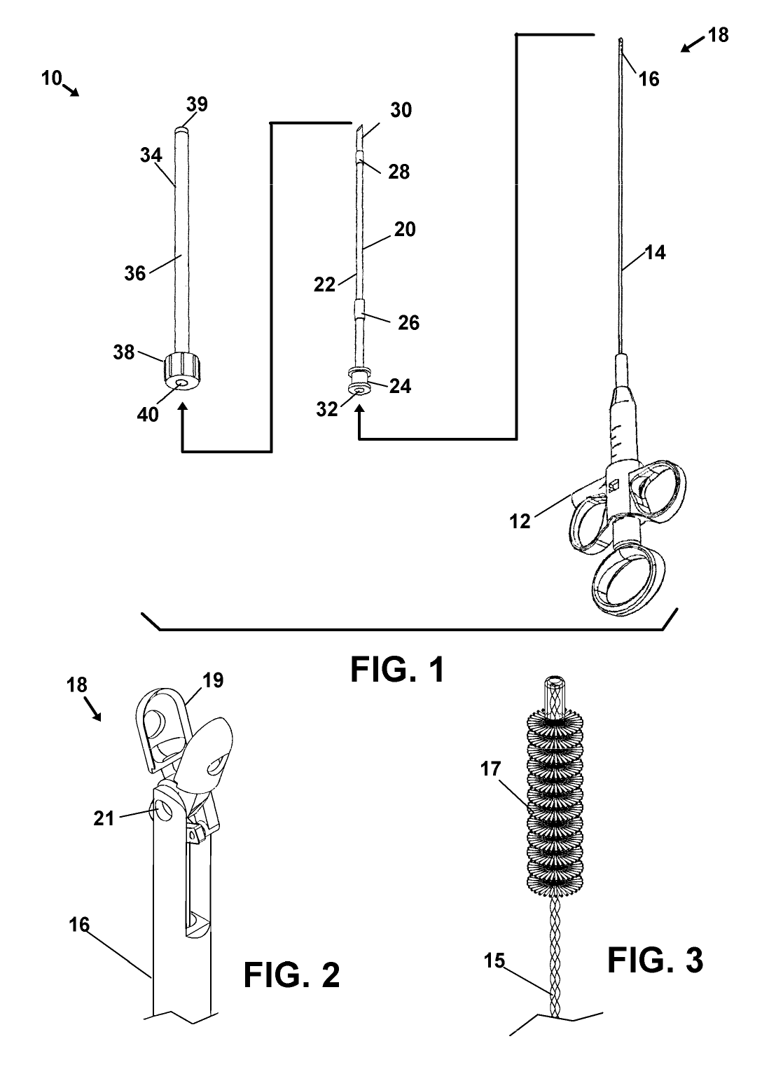

Now referring to drawings in FIGS. 1-12, wherein similar components are identified by like reference numerals, there is seen in FIG. 1 a view showing components of a particularly preferred mode of the tissue sampling device 10. As shown, there is a disassembled view of the device 10 which includes at least a combination of an aspiration needle 30 and biopsy forceps 18. The device 10 includes biopsy forceps 18 having a handle 12 and elongated flexible member such as the depicted control wire 14 extending to a distal end 16. Located at the distal end 16 is the forceps jaws 19 (FIG. 2) which are employable for tissue and/or cell sampling at a shallow or deep tissue position depending on the use by the surgeon. The handle 12 is engaged to the control wire 14 which provides a means to manipulate the jaws 19 of the forceps 18 by translation of the control wire 14.

The needle assembly 20 is formed and translated using another flexible member the depicted supporting shaft member 22 extending from a handled end 24 to a aspiration needle 30 positioned at the distal end. The shaft member 22 has a hollow axial passageway or axial passage defined by a sidewall of the shaft member 22 and is preferably made from a flexible material with a low coefficient of friction such as Teflon as a means to enhance translation and minimize friction when translatabley engaged within a lumen such as that of a catheter. However, it can be formed from any polymeric or metallic material employable in the art of medical instruments. The aspiration needle 30 is engaged to the shaft 22 via a crimp 28. However, it can be engaged by any means known in the art such as adhesive, collaring, cooperating fasteners, or other operative means of engagement as would occur to those skilled in the art.

The axial passageway or axial passage 32 communicates along the length of the assembly 20 from an open end at the handle 32, to an open exiting end, lining up the axial passageway with the hollow core of the needle 30 where the passageway or passage 32 extends through the hollow core of the aspiration needle 30 to form a continuous axial passageway. In use, and shown in later figures, the elongated control wire 14 communicating to control the forceps 18 is translatably and coaxially engaged within the passage 32 such as to position the jaws 19 at or near a position within or surrounded by the wall defining the hollow axial core of the aspiration needle 30. When so surrounded, the forceps 18 bearing a tissue and/or cell sample, are protected during removal, from both the tissue and the lumen, by the wall forming the aspiration needle, which provides a means to protect the sample from loss and contamination. The needle assembly 20 additionally includes a stop member 26.

Additionally shown, is an exterior sleeve component 34 having an elongated shaft member 36 engaged to a circular handled end 38. The sleeve component 34 has an axial passage 40 communicating therethrough from an open end at the handle 38 to an open distal end 39. Shown in more detail in subsequent drawings, the sleeve component 34 is translatably and coaxially engaged to encircle the needle assembly 20 and the forceps 18 which are translatable within the axial passage 40 of the sleeve component 34.

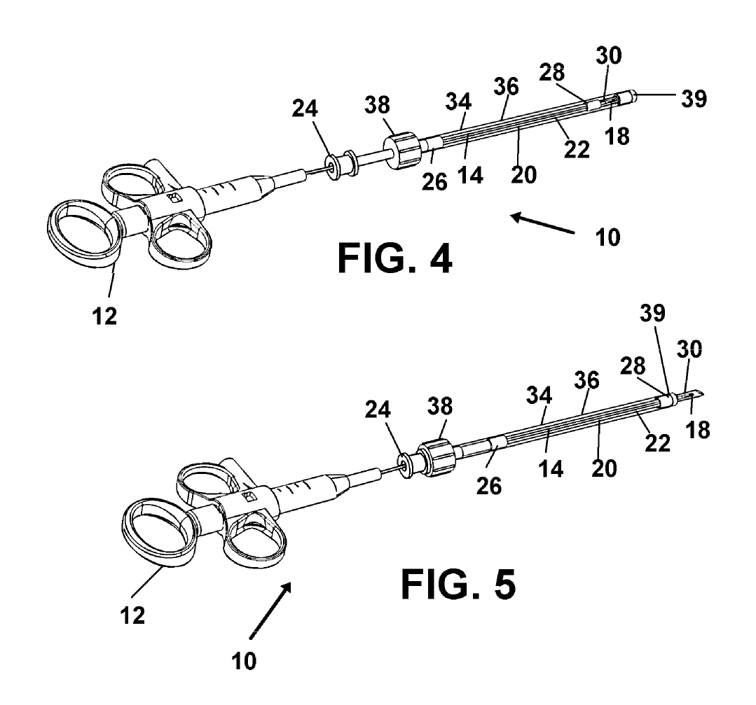

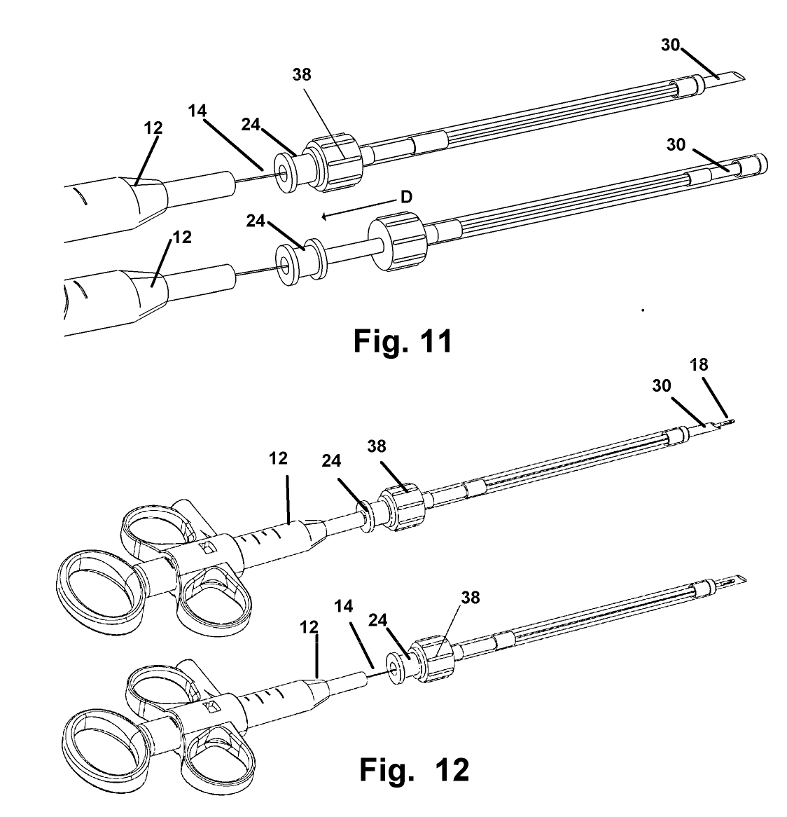

In use, shown in (FIGS. 4-6 and 11-12) the stop member 26 restricts the translational movement of the sleeve component 34 relative to the needle assembly 20. The stop member 26 also restricts the distance of translation “D” of FIG. 11, of the needle assembly 20 relative to the sleeve component 34, to the length of the needle assembly 20 between the handle 38 and the stop member 26.

It must be noted that it is within the scope of the invention that the device 10 may be modified in form to be longer, shorter, or to achieve other structural configurations which will allow a physician to obtain deep tissue sampling through translation of the aspiration needle 30 and forceps 18 coaxial to a surrounding sleeve component 34. As such those skilled in the art will appreciate that the depictions set forth are provided merely for descriptive purposes to portray the overall intent and scope of the invention, and should not be considered limiting. Additionally, the device is capable of achieving the noted goals of obtaining deep tissue and/or cell samples which are protected during retrieval through means other then biopsy forceps 18, such as the employment of a cytology brush 17 (FIG. 3)which is translatable to gather cells from deep tissue positions in the same fashion as the forceps 18.

FIG. 2 shows a view of the distal end 16 of the forceps 18 showing the jaws 19 employed for grasping, cutting, and retrieving tissue and/or cell samples from deep tissue positions or at the option of the user, from shallow or surface positions. In use, the aspiration needle 30 penetrates the tissue at the determined sampling site. In doing so, the needle 30 provides a means to tunnel into the tissue and/or cell sampling site and creates an elongated passage or a seam through which the forceps 18 are translated in a second sequential action to retrieve tissue or cells from the deep end of the bored tunnel or tunnel seam into the tissue sampling site. If used separately, the needle 30, can on its own, remove a portion of tissue. However, such can be small and indeterminate as to actual location.

Once the tunnel is formed, the forceps 18 are translated past the distal end of the needle 30 where the jaws 19 can extend and be translated into the seam formed by the needle 30 or a tunnel formed by the needle 30 depending upon the manner in which the needle 30 is employed in the first action of the sequential sampling. Once at the deeper position provided by the seam formed by the wall of the needle 30 or the tunnel formed by the coring of the needle 30, the jaws 19 may be contracted by the controller to retrieve a large tissue and/or cell sample from a deep tissue location which is at the distal end of the needle 30. Sample cutting is accomplished through translation of the control wire 14 by translation of the handle 12. This is of an advantage over prior art in which conventionally forceps 18 are limited to only grasping surface tissue at the sampling site.

In the device herein used for deep tissue and/or cell sampling, the forceps 18 are projected past the distal end of the operatively positioned needle 30, which has formed a tunnel ending at its distal end, to the known deep tissue position. At that point, deep in the tissue sampling site, a piece of tissue is excised, and the forceps 18 are thereafter translated back within the cavity formed by the wall of the needle 30 to a retracted position. In this position, the tissue and/or cell sample is protected from loss and contamination by other tissues during retrieval of the needle and forceps from the patient. Thus, a large sample, from a known position deep in the tissue at the sampling site, is retrieved and protected during the retrieval process.

As is shown, the jaws 19 are engaged about a hinge 21 providing a lever to manipulate the jaws 19 during translation of the control wire 14. It is preferred that the handle 12 of the forceps 18 provide the means to manipulate the jaws 19 however in other modes of the device 10, the forceps 18 may be manipulated remotely by a robot or by other means as would occur to those skilled in the art.

FIG. 3 shows a view of another particularly preferred mode of the device 10 wherein a cytology brush 17 is substituted for the forceps 18 and employed for deep tissue sampling. In this mode, the guide control wire 14 extending from the handle 12 is shown in FIG. 3 as guidewire 15 in communication with the brush end 17. The slight modifications necessary to employ the cytology brush 17 or other tissue sampling means that one skilled in the art would immediately recognize are considered within the scope of the invention and are anticipated. Further, the cytology brush 17 and the forceps 18 can have an engagement means on the control wire 14 or guidewire 15 allowing them to be interchanged and they may be provided in a kit to allow the medical professional to engage either to the control wire 14. Threaded engagement between the two components is one mode using a threaded cavity and threaded guide wire or control wire to engage the cytology brush 17 or forceps 18.

Referring now to FIG. 4-FIG. 6, there is shown preferred operative steps of employment of the device 10 employing forceps 18. Again, it is noted that the device in other modes, employing other means for tissue sampling, would follow the same general operative procedure during use, and is anticipated.

In FIG. 4, there is shown the assembled device 10 with the needle assembly 20 engaged and translated to surround the forceps guide control wire 14 and the protective sleeve 34 engaged thereover as well with all components coaxial. The concentric coaxial translational engagement of the components allow the aspiration needle 30 and forceps 18 to be translatabley positionable to respective extended or as-used positions and intermediate positions as is shown in FIGS. 4-6 and 11-12 for example.

The device 10 in FIG. 4 is shown in a retracted or stored position with the aspiration needle 30 and forceps 18 both retracted into the distal end 39 of the protective sleeve 34 provided by shaft member 36. Further, as can be seen, this position is accomplished by translating the protective sleeve 34 away from the handles 12 and 24 until the handle 38 of the sleeve 34 engages the stop element 26 on the needle assembly 20.

The device 10 is positionable to place the aspiration needle 30 in an extended position as shown in FIG. 5 by actuation of the controller engaged to the needle assembly 20 where it may be employed to form a tunnel, or an elongated incision or seam formed by the wall of the needle 30, through which the forceps 18 may be subsequently translated by manipulation. The flexible member or control wire 14 may be manipulated by a controller to translate into the tunnel or seam, and then actuated to have the jaws 19 remove a tissue portion in a deep tissue and/or cell sampling.

As also depicted, the forceps 18 are maintained in the stored position within the axial passage 32 of the aspiration needle 30. This is accomplished by translating the protective sleeve element 34 towards the handle 24 of the needle assembly 20 to expose the aspiration needle 30 from the open distal end 39 or by translation of the wire 14 to move the forceps 18 to the retracted position where the tissue or cell sample is protected during retrieval of the needle 30 and the forceps 18. The manner of control of both of the needle 30 and the forceps 18 of course can be with any mode of a first flexible member for the needle 30 and a second flexible member for the forceps 18 so long as the there is an axial passageway along the first flexible member to allow the second flexible member to operate, translate, and actuate the forceps 18 through it.

As can be seen, translating to this position, the handle 24 of the needle assembly 20 acts as a stopper for the handle 38 of the sleeve 34. This position allows the physician or surgeon to insert the aspiration needle 30 to form the tunnel at the tissue sampling site to the desired depth into tissue to be sampled. Thereafter, from the known position at the distal end of the aspiration needle 30, tissue may be removed using the forceps 18 or cytology brush. Once a sample is taken, the forceps 18 or brush may be translated back within the safe confines of the cavity surrounded by the wall of the needle 30. Those skilled in the art will realize that it is within the scope of the invention that the wall of the aspiration needle 30 may be shorter or longer than the depiction as needed to obtain the desired tunnel depth, and such is anticipated within the scope of this invention.

As noted, once inserted, the aspiration needle 30 creates the tunnel in the tissue to the deep sampling site where the forceps 18 are extended through translation of the control wire 14. From the known deep tissue sampling position, the forceps 18 may cut and retrieve a portion of tissue.

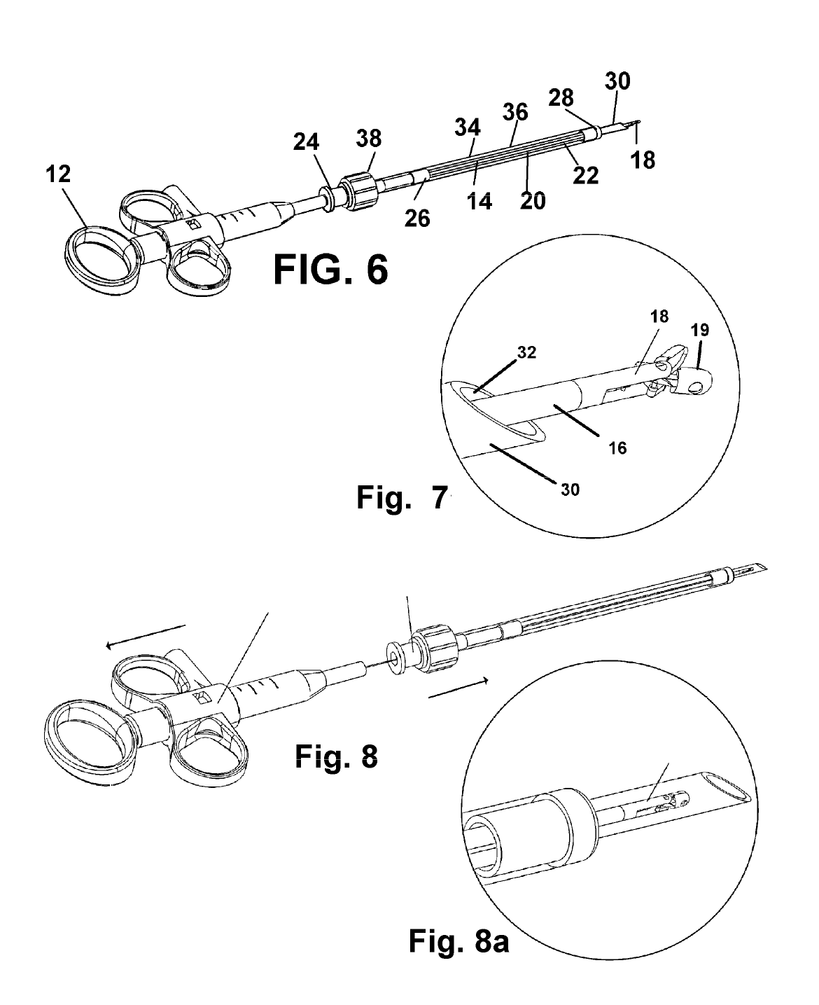

FIG. 6 depicts a view of the device 10 with the forceps 18 in the extended or as-used tissue excising or sampling position, with the forceps 18 extended past the opening at the distal end of the aspiration needle 30 as shown. This is accomplished by translating the needle assembly 20 and sleeve 34 concurrently toward the forceps handle 12 thereby translating the control wire 14 relative thereto, such that the needle component handle 24 is in an abutment with the distal end of the forceps handle 12 as shown. It must be noted that it is within the scope of the device 10 to employ a longer control wire 14 as to allow the forceps 18 to be translated further into the deep tissue sampling position, and such is anticipated. The forceps 18 are operated in a conventional fashion to take a slice of tissue and once tissue is retrieved and between the jaws 19 of the forceps 18, the aspiration needle 30 and forceps 18 can be translated back to the stored position.

FIGS. 7 and 7a shows an enlarged view of forceps 18 in the extended position of FIG. 6. Again, the forceps 18 extend from the axial passage 32 of the needle assembly 20 and are surrounded by the wall of the aspiration needle 30. As is shown, the jaws 19 of the forceps 18 are in an open position as would be accomplished by manipulation by the forceps handle 12.

FIG. 8 depicts the device 10 in which the aspiration needle 30 is in the extended position and FIG. 8a shows an enlarged view of the forceps 18 in the retracted position within the aspiration needle 30.

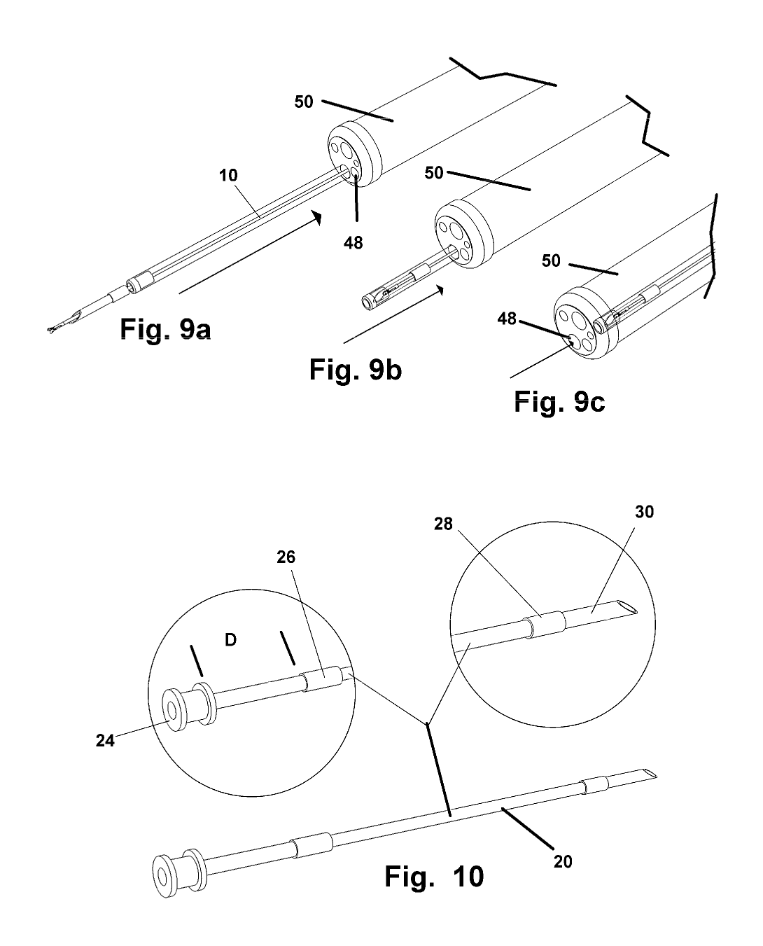

FIGS. 9a-9c depict the device 10 translating within a lumen, for example, a lumen 48 formed in a bronchoscope 50 employed for a lung tissue biopsy. Use in any elongated lumen bearing device is anticipated, however.

FIG. 10 depicts components forming an inner tubular structure of the needle assembly 20 which translates within a surrounding outer tubular structure of the protective sleeve 34. The aspiration needle 30 is shown at the distal end.

FIG. 11 depicts a translation of the translating needle assembly 20, within the coaxial surrounding protective sleeve 34 used to translate the aspiration needle 30 between a retracted position of FIG. 4 and the extended position of FIG. 5.

FIG. 12 depicts the needle assembly 20 translated to the extended position and the result of translation of a control wire 14 to translate the forceps 18 engaged thereon, to the extended position such as in FIG. 7a.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. A tissue and/or cell sampling device, comprising:
a catheter, said catheter being flexible and having a lumen communicating between a proximal end and a distal end;
a first flexible control member having a first end and a second end, said control member communicating through said lumen from said proximal end to an engagement at said second end with a proximal end of a needle;
said needle having an axial conduit communicating therethrough from said proximal end to a distal end opposite said proximal end;
an axial pathway communicating through said first flexible control member and said axial conduit of said needle to said distal end of said needle;
a tissue and/or cell sampling component translatabley positioned within said axial conduit;
said tissue and/or cell sampling component translatable from a retraced position within said axial conduit to an extended position extending past said distal end of said needle;
a second control member communicating from a first end through said axial pathway to an engagement at a second end with said tissue and/or cell sampling component;
said hollow needle operable to dislodge a core of tissue through a translation of said first control member; and
said tissue and/or cell sampling component positionable between said retracted position and said extended position through a manipulation of said second control member;
said tissue and/or cell sampling component in said extended position operable to remove a portion of said tissue, and move to said retracted position with said tissue protected within said axial conduit, whereby either or both of said hollow needle and said tissue and/or cell sampling component are employable for retrieving samples of said tissue.

2. The tissue and/or cell sampling device of claim 1 additionally comprising:
said needle and said tissue and/or cell sampling component employable in a sequence; and
a first act of said sequence dislodging said tissue core; and
said tissue and/or cell sampling component removing said portion of said tissue from a distal end of said tissue core, whereby said portion of said tissue is retrievable from a deeper position in said tissue being sampled than said distal end of said core.

3. The tissue and/or cell sampling device of claim 1 additionally comprising:
said tissue sampling component being a pair of forceps.

4. The tissue and/or cell sampling device of claim 1 additionally comprising:
said tissue and/or cell sampling component being a brush.

5. The tissue and/or cell sampling device of claim 2 additionally comprising:
said tissue and/or cell sampling component being a pair of forceps.

6. The tissue and/or cell sampling device of claim 2 additionally comprising:
said tissue sampling component being a brush.

7. The tissue and/or cell sampling device of claim 1 additionally comprising:
a controller engaged to said first end of said first control member, said controller configured for user manipulation to remotely induce said translation of said first control member.

8. The tissue and/or cell sampling device of claim 1 additionally comprising:
a second controller engaged to said first end of said second control member;
a manipulation of said second controller providing movement of said second control member to position said tissue and/or cell sampling component between said retracted position and said extended position; and
a second manipulation of said second controller communicating an actuation of said tissue and/or cell sampling component in said extended position, to cause a removing of said portion of said tissue.

9. The tissue and/or cell sampling device of claim 8 additionally comprising:
a second controller engaged to said first end of said second control member;
a manipulation of said second controller providing movement of said second control member to position said tissue and/or cell sampling component between said retracted position and said extended position; and
a second manipulation of said second controller communicating an actuation of said tissue sampling component in said extended position, to cause a removing of said portion of said tissue.

10. The tissue and/or cell sampling device of claim 2 additionally comprising:
a controller engaged to said first end of said first control member, said controller configured for user manipulation to remotely induce said translation of said first control member.

11. The tissue and/or cell sampling device of claim 2 additionally comprising:
a second controller engaged to said first end of said second control member;
a manipulation of said second controller providing movement of said second control member to position said tissue and/or cell sampling component between said retracted position and said extended position; and
a second manipulation of said second controller communicating an actuation of said tissue and/or cell sampling component in said extended position, to cause a removing of said portion of said tissue.

12. The tissue and/or cell sampling device of claim 10 additionally comprising:
a second controller engaged to said first end of said second control member;
a manipulation of said second controller providing movement of said second control member to position said tissue and/or cell sampling component between said retracted position and said extended position; and
a second manipulation of said second controller communicating an actuation of said tissue and/or cell sampling component in said extended position, to cause a removing of said portion of said tissue.

13. The tissue and/or cell sampling device of claim 5 additionally comprising:
a controller engaged to said first end of said first control member, said controller configured for user manipulation to remotely induce said translation of said first control member.

14. The tissue and/or cell sampling device of claim 5 additionally comprising:
a second controller engaged to said first end of said second control member;
a manipulation of said second controller providing movement of said second control member to position said tissue sampling component between said retracted position and said extended position; and
a second manipulation of said second controller communicating an actuation of said tissue sampling component in said extended position, to cause a removing of said portion of said tissue.

15. The tissue and/or cell sampling device of claim 13 additionally comprising:
a second controller engaged to said first end of said second control member;
a manipulation of said second controller providing movement of said second control member to position said tissue sampling component between said retracted position and said extended position; and
a second manipulation of said second controller communicating an actuation of said tissue and/or cell sampling component in said extended position, to cause a removing of said portion of said tissue.

16. The tissue and/or cell sampling device of claim 6 additionally comprising:
a controller engaged to said first end of said first control member, said controller configured for user manipulation to remotely induce said translation of said first control member.

17. The tissue and/or cell sampling device of claim 6 additionally comprising:
a second controller engaged to said first end of said second control member;
a manipulation of said second controller providing movement of said second control member to position said tissue and/or cell sampling component between said retracted position and said extended position; and
a second manipulation of said second controller communicating an actuation of said tissue and/or cell sampling component in said extended position, to cause a removing of said portion of said tissue.

18. The tissue and/or cell sampling device of claim 16 additionally comprising:
a second controller engaged to said first end of said second control member;
a manipulation of said second controller providing movement of said second control member to position said tissue and/or cell sampling component between said retracted position and said extended position; and
a second manipulation of said second controller communicating an actuation of said tissue and/or cell sampling component in said extended position, to cause a removing of said portion of said tissue.
